(19) United States
(12) Patent Application Publication
(10) Pub. No.: US 2012/0226141 A1
(43) Pub. Date: Sep. 6, 2012

(54) MAGNETIC RESONANCE IMAGING APPARATUS AND MAGNETIC RESONANCE IMAGING METHOD

(75) Inventors: Kensuke SHINODA, Otawara-shi (JP); Satoshi WAKAI, Nasushiobara-shi (JP)

(73) Assignees: TOSHIBA MEDICAL SYSTEMS CORPORATION, OTAWARA-SHI (JP); KABUSHIKI KAISHA TOSHIBA, TOKYO (JP)

(21) Appl. No.: 13/399,178

(22) Filed: Feb. 17, 2012

(30) Foreign Application Priority Data
Mar. 2, 2011 (JP) 2011-045233
Jan. 24, 2012 (JP) 2012-011600

(51) Int. Cl. A61B 5/055

(52) U.S. Cl. 600/419; 600/410

(57) ABSTRACT

According to one embodiment, a magnetic resonance imaging apparatus includes a structural information acquisition unit, an abnormal part detection unit, an imaging region setting unit and an imaging unit. The structural information acquisition unit is configured to acquire anatomical structural information based on first image data of an object. The abnormal part detection unit is configured to detect an abnormal region based on the structural information. The imaging region setting unit is configured to indicate an imaging region according to a detection result of the abnormal region. The imaging unit is configured to acquire second image data of the object by imaging of an imaging region set based on the imaging region according to the detection result of the abnormal region.

### CROSS REFERENCES TO RELATED APPLICATIONS

This application is based upon and claims the benefit of priorities from Japanese Patent Application No. 2011-45233 filed on Mar. 2, 2011 and Japanese Patent Application No. 2012-11600 filed on Jan. 24, 2012; the entire contents of Japanese Patent Application No. 2011-45233 and Japanese Patent Application No. 2012-11600 are incorporated herein by reference.

### FIELD

Embodiments described herein relate generally to a MRI (magnetic resonance imaging) apparatus and a magnetic resonance imaging method.

### BACKGROUND

MRI is an imaging method which excites nuclear spin of an object set in a static magnetic field with a RF (radio frequency) signal having the Larmor frequency magnetically and reconstructs an image based on MR (magnetic resonance) signals generated due to the excitation.

MRI can obtain images having mutually different contrasts by changing imaging conditions. Therefore, MRI is considered to be useful for estimating a nature of plaque in a blood vessel. Especially, a carotid branches into an inner carotid and an outer one from a gross carotid. Hence, plaque easily gathers around a branch part. Plaque is stable or unstable. Plaque gathering in a blood vessel causes cerebral infarction. Therefore, estimating a nature of plaque is important.

For estimating a nature of plaque, a feature of blood vessels such as a carotid is depicted by MRA (magnetic resonance angiography) such as TOF (time of flight) method firstly. Next, an operator observes feature images of the blood vessels to specify an abnormal part such as a stenosis. Subsequently, the operator sets imaging regions involving the stenotic part. Then, section images and/or axial images of blood vessels around the stenotic part are acquired for examination of a nature of the plaque.

On the other hand, cine image of the stenotic part is acquired for obtaining flow velocities of blood flow around the stenotic part. by an imaging method such as PS (phase shift) flow method. In case of measuring flow velocities of blood flow, it is necessary to set imaging sections normal to a direction of the blood flow for measuring the flow velocities with the highest accuracy theoretically.

Setting of imaging conditions for estimation of a nature of plaque by MRI is desired to be easier. That is, it is desired to make it possible to set an imaging region involving a stenotic part easily. In addition, it is desired to make it possible to set sections normal to a traveling direction of blood vessel as imaging regions easily for measuring flow velocities of the blood flow with higher accuracy.

This requirement is common to MRA having a purpose other than estimation of a nature of plaque. Further, the same applies to imaging of a target, such as tissue or an organ, other than a blood vessel.

An object of the present invention is to provide a magnetic resonance imaging apparatus and a magnetic resonance imaging method which allow to set an imaging region for imaging a diseased part more easily.

### BRIEF DESCRIPTION OF THE DRAWINGS

In the accompanying drawings:

FIG. 1 is a block diagram showing a magnetic resonance imaging apparatus according to an embodiment of the present invention;

FIG. 2 is a functional block diagram of the computer shown in FIG. 1;

FIG. 3 is a flowchart showing a flow for plaque imaging of a carotid artery of an object by the magnetic resonance imaging apparatus as shown in FIG. 1;

FIG. 4 is a view showing an example of center line, outline and junction of carotid arteries obtained by the structural information acquiring part shown in FIG. 2;

FIG. 5 is a view for explaining the first example method of setting imaging slice sections by the imaging section calculation part shown in FIG. 2;

FIG. 6 is a view for explaining the second example method of setting imaging slice sections by the imaging section calculation part shown in FIG. 2;

FIG. 7 is a view for explaining the third example method of setting imaging slice sections by the imaging section calculation part shown in FIG. 2;

FIG. 8 is a view showing an example of overlaying and displaying imaging slice sections on a VR image of blood vessels for adjusting the imaging slice sections by the imaging section correction part shown in FIG. 2;

FIG. 9 is a view for explaining a method for moving an imaging slice section along the center line of the blood vessel by the imaging section correction part shown in FIG. 2;

FIG. 10 is a view showing an example of overlaying and displaying imaging slice sections on a SPR image of blood vessels for adjusting the imaging slice sections by the imaging section correction part shown in FIG. 2; and

FIG. 11 is a view showing a plaque region to be a target of the plaque imaging by the magnetic resonance imaging apparatus shown in FIG. 1.

### DETAILED DESCRIPTION

In general, according to one embodiment, a magnetic resonance imaging apparatus includes a structural information acquisition unit, an abnormal part detection unit, an imaging region setting unit and an imaging unit. The structural information acquisition unit is configured to acquire anatomical structural information based on first image data of an object. The abnormal part detection unit is configured to detect an abnormal region based on the structural information. The imaging region setting unit is configured to indicate an imaging region according to a detection result of the abnormal region. The imaging unit is configured to acquire second image data of the object by imaging of an imaging region set based on the imaging region according to the detection result of the abnormal region.

In addition, a magnetic resonance imaging apparatus according to an embodiment of the present invention includes a center line information acquisition unit, an indication unit, a confirmation unit and an imaging unit. The center line information acquisition unit is configured to acquire center line information of an imaging target based on first image data of an object. The indication unit is configured to indicate a pick of an abnormal region and an orthogonal section for imaging the pick of the abnormal region based on the center line information. The confirmation unit is configured to confirm an imaging section with referring to the indicated orthogonal section. The imaging unit is configured to acquire second image data of the object by imaging of the imaging section.

In addition, a magnetic resonance imaging method according to an embodiment of the present invention includes acquiring anatomical structural information based on first image data of an object; detecting an abnormal region based on the structural information; indicating an imaging region according to a detection result of the abnormal region; and acquiring second image data of the object by imaging of an imaging region set based on the imaging region according to the detection result of the abnormal region.

A magnetic resonance imaging apparatus and a magnetic resonance imaging method according to embodiments of the present invention will be described with reference to the accompanying drawings.

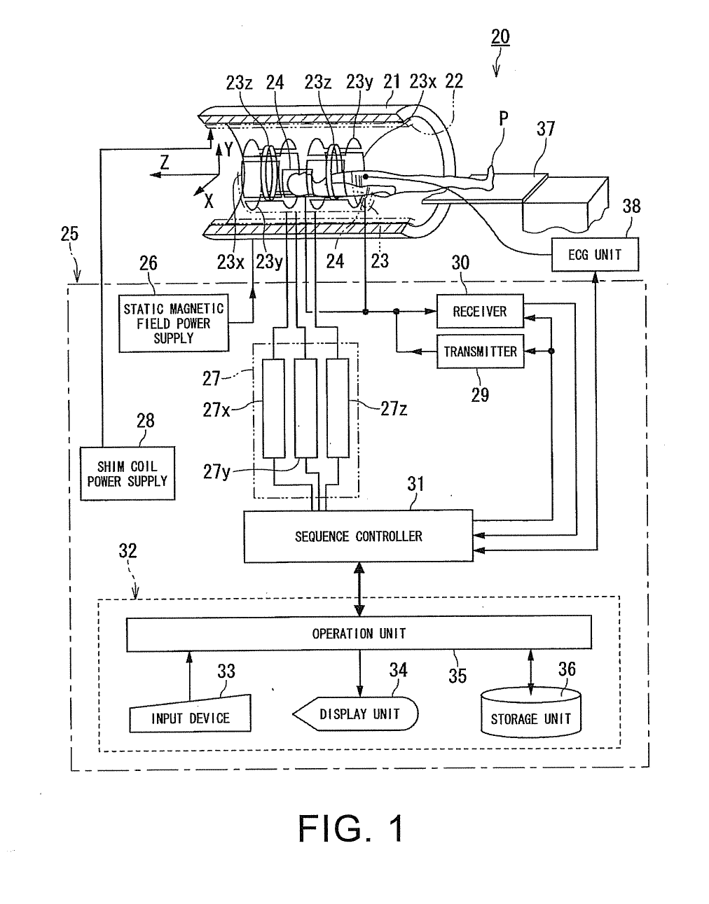

FIG. 1 is a block diagram showing a magnetic resonance imaging apparatus according to an embodiment of the present invention.

A magnetic resonance imaging apparatus 20 includes a static field magnet 21 for generating a static magnetic field, a shim coil 22 arranged inside the static field magnet 21 which is cylinder-shaped, a gradient coil 23 and RF coils 24.

The magnetic resonance imaging apparatus 20 also includes a control system 25. The control system 25 includes a static magnetic field power supply 26, a gradient power supply 27, a shim coil power supply 28, a transmitter 29, a receiver 30, a sequence controller 31 and a computer 32. The gradient power supply 27 of the control system 25 includes an X-axis gradient power supply 27x, a Y-axis gradient power supply 27y and a Z-axis gradient power supply 27z. The computer 32 includes an input device 33, a display unit 34, an operation unit 35 and a storage unit 36.

The static field magnet 21 communicates with the static magnetic field power supply 26. The static magnetic field power supply 26 supplies electric current to the static field magnet 21 to get the function to generate a static magnetic field in a imaging region. The static field magnet 21 includes a superconductivity coil in many cases. The static field magnet 21 gets current from the static magnetic field power supply 26 which communicates with the static field magnet 21 at excitation. However, once excitation has been made, the static field magnet 21 is usually isolated from the static magnetic field power supply 26. The static field magnet 21 may include a permanent magnet which makes the static magnetic field power supply 26 unnecessary.

The static field magnet 21 has the cylinder-shaped shim coil 22 coaxially inside itself. The shim coil 22 communicates with the shim coil power supply 28. The shim coil power supply 28 supplies current to the shim coil 22 so that the static magnetic field becomes uniform.

The gradient coil 23 includes an X-axis gradient coil 23x, a Y-axis gradient coil 23y and a Z-axis gradient coil 23z. Each of the X-axis gradient coil 23x, the Y-axis gradient coil 23y and the Z-axis gradient coil 23z which is cylinder-shaped is arranged inside the static field magnet 21. The gradient coil 23 has also a bed 37 in the area formed inside it which is an imaging area. The bed 37 supports an object P. The RF coils 24 include a whole body coil (WBC: whole body coil), which is built in the gantry, for transmission and reception of RF signals and local coils, which are arranged around the bed 37 or the object P, for reception of RF signals.

The gradient coil 23 communicates with the gradient power supply 27. The X-axis gradient coil 23x, the Y-axis gradient coil 23y and the Z-axis gradient coil 23z of the gradient coil 23 communicate with the X-axis gradient power supply 27x, the Y-axis gradient power supply 27y and the Z-axis gradient power supply 27z of the gradient power supply 27 respectively.

The X-axis gradient power supply 27x, the Y-axis gradient power supply 27y and the Z-axis gradient power supply 27z supply currents to the X-axis gradient coil 23x, the Y-axis gradient coil 23y and the Z-axis gradient coil 23z respectively so as to generate gradient magnetic fields Gx, Gy and Gz in the X, Y and Z directions in the imaging area.

The RF coils 24 communicate with the transmitter 29 and/or the receiver 30. The transmission RF coil 24 has a function to transmit a RF signal given from the transmitter 29 to the object P. The reception RF coil 24 has a function to receive a MR signal generated due to an nuclear spin inside the object P which is excited by the RF signal to give to the receiver 30.

The sequence controller 31 of the control system 25 communicates with the gradient power supply 27, the transmitter 29 and the receiver 30. The sequence controller 31 has a function to storage sequence information describing control information needed in order to make the gradient power supply 27, the transmitter 29 and the receiver 30 drive and generate gradient magnetic fields Gx, Gy and Gz in the X, Y and Z directions and a RF signal by driving the gradient power supply 27, the transmitter 29 and the receiver 30 according to a predetermined sequence stored. The control information above-described includes motion control information, such as intensity, impression period and impression timing of the pulse electric current which should be impressed to the gradient power supply 27

The sequence controller 31 is also configured to give raw data to the computer 32. The raw data is complex data obtained through the detection of a MR signal and A/D (analog to digital) conversion to the MR signal detected in the receiver 30.

The transmitter 29 has a function to give a RF signal to the RF coil 24 in accordance with control information provided from the sequence controller 31. The receiver 30 has a function to generate raw data which is digitized complex number data by detecting a MR signal given from the RF coil 24 and performing predetermined signal processing and A/D converting to the MR signal detected. The receiver 30 also has a function to give the generated raw data to the sequence controller 31.

In addition, an ECG (electro cardiogram) unit 38 for acquiring an ECG signal of the object P is provided with the magnetic resonance imaging apparatus 20. The ECG signal detected by the ECG unit 38 is outputted to the computer 32 through the sequence controller 31. Then, an ECG gated imaging for acquiring MR signals in synchronized with an ECG signal can be performed, as needed.

Note that, a PPG (peripheral pulse gating) signal representing a beat as pulse wave information may be acquired instead of an ECG signal representing a beat as heart rate information. A PPG signal is acquired by detecting a pulse wave of e.g. tip of a finger as an optical signal. When a PPG signal is acquired, a PPG signal detection unit is provided with the magnetic resonance imaging apparatus 20.

The computer 32 gets various functions by the operation unit 35 executing some programs stored in the storage unit 36 of the computer 32. Alternatively, some specific circuits having various functions may be provided with the magnetic resonance imaging apparatus 20 instead of using some of the programs.

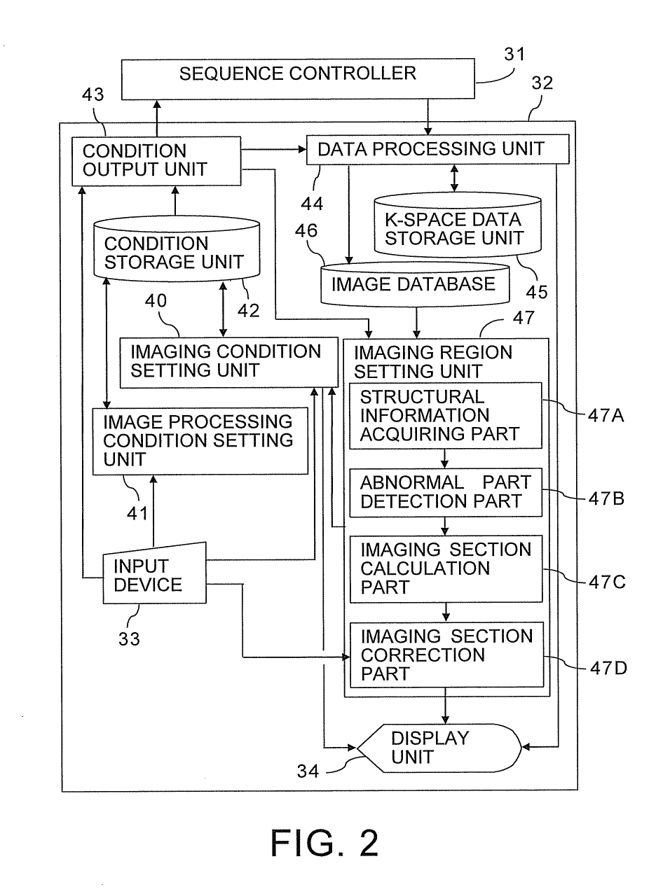

FIG. 2 is a functional block diagram of the computer 32 shown in FIG. 1.

The computer 32 functions as an imaging condition setting unit 40, an image processing condition setting unit 41, a condition storage unit 42, a condition output unit 43, a data processing unit 44, a k-space data storage unit 45, an image database 46 and an imaging region setting unit 47 by executing the programs stored in the storage unit 36. Further, the imaging region setting unit 47 has a structural information acquiring part 47A, an abnormal part detection part 47B, an imaging section calculation part 47C and the imaging section correction part 47D.

The imaging condition setting unit 40 has a function to set imaging conditions including a pulse sequence based on instruction for setting the imaging conditions inputted from the input device 33 and a function to write the set imaging conditions to the condition storage unit 42. Note that, the imaging condition setting unit 40 is configured to acquire past imaging conditions from the condition storage unit 42 and display the past imaging conditions together with a screen for setting imaging conditions on the display unit 34 so that the past imaging conditions can be referred and edited in order to set imaging conditions.

In addition, when the imaging region setting unit 47 has supplied setting information of imaging regions to the imaging condition setting unit 40, the imaging condition setting unit 40 is configured to set imaging regions set by the imaging region setting unit 47 as imaging regions for an imaging scan.

The image processing condition setting unit 41 has a function to set image processing conditions such as subtraction processing to image data having been obtained by imaging based on instruction for setting the image processing conditions inputted from the input device 33 and a function to write the set image processing conditions in the condition storage unit 42. Note that, the image processing condition setting unit 41 is configured to acquire past image processing conditions from the condition storage unit 42 so that the past image processing conditions can be referred in order to set image processing conditions.

The condition output unit 43 has a function to output imaging conditions acquired from the condition storage unit 42 to the sequence controller 31 so as to control the sequence controller 31 to perform a scan and output the imaging conditions and image processing conditions acquired from the condition storage unit 42 to the data processing unit 44, according to control instruction such as instruction for starting imaging or stopping imaging inputted from the input device 33. In addition, the condition output unit 43 has a function to supply the imaging conditions and the image processing conditions acquired from the condition storage unit 42 to the imaging region setting unit 47 according to instruction inputted from the input device 33.

The data processing unit 44 has a function to acquire MR echo signals from the sequence controller 31 to arrange the acquired MR echo signals in the k-space formed in the k-space data storage unit 45 as k-space data, a function to read k-space data from the k-space data storage unit 45 to perform image reconstruction processing including FT (Fourier transform) and image processing based on image processing conditions acquired from the condition output unit 43 so as to generate diagnosis data such as image data or flow velocities of blood flow, a function to write image data or diagnosis data to the image database 46 and a function to perform necessary image processing of diagnosis data or image data acquired from the image database 46 to display the processed data on the display unit 34. Note that, the data processing unit 44 is configured to add imaging conditions acquired from the condition output unit 43 and corresponding patient information to image data or diagnosis data as incidental information.

The imaging region setting unit 47 has a function to acquire image data usable for setting imaging regions from the image database 46 and set appropriate imaging regions based on imaging conditions and image processing conditions acquired from the condition output unit 43 and the acquired image data. Note that, the imaging region setting unit 47 may be configured to indicate options for imaging regions prior to setting of the imaging regions and set the imaging region according to confirmation information inputted from the input device 33. In this case, plural options for imaging regions may be indicated so that the imaging regions are set according to selection information inputted from the input device 33.

In addition, the imaging region setting unit 47 has a function to correct imaging sections automatically calculated as imaging regions once according to information inputted from the input device 33. Then, the imaging region setting unit 47 is configured to supply the set imaging regions as setting information thereof to the imaging condition setting unit 40.

The structural information acquiring part 47A has a function to acquire anatomical structural information of tissue and/or organs, such as an outline, center lines and junctions of blood vessels or an outline and a center line of a spine, from image data by data analysis processing according to imaging conditions and image processing conditions acquired from the condition output unit 43. For that purpose, the structural information acquiring part 47A stores information necessary for the data analysis processing such as known anatomical information of human body

By the structural information acquiring part 47A, plural pieces of structural information with regard to a desired organ such as a blood vessel or a spine can be acquired. For example, when a target of which structural information is to be acquired is a blood vessel, a center line and an inner cavity of the blood vessel can be acquired as the first and the second pieces of structural information respectively. However, at least one of the center line and the inner cavity of the blood vessel may be acquired as structural information of the blood vessel.

In order to perform data processing for acquiring these pieces of structural information, arbitrary processing such as edge extraction processing or pattern matching processing to anatomical information of human body can be used according to a purpose.

The abnormal part detection part 47B has a function to detect a position and a range of a part of which feature is abnormal, such as blood vessel stenosis, as abnormal part region information by data analysis processing based on structural information of tissue and/or organs. Note that, abnormal part region information can be detected from anatomical structural information acquired by the structural information acquiring part 47A. However, abnormal part region information can be also detected from image data referred to acquire the anatomical structural information by the structural information acquiring part 47A.

The imaging section calculation part 47C has a function to calculate sizes, positions and directions of imaging slice sections as imaging regions automatically by data processing based on abnormal part region information. Imaging regions can be automatically calculated as regions covering an abnormal region appropriately. In addition, imaging regions may be automatically calculated with referring to structural information of tissue and/or organs and image data. The automatically calculated imaging regions can be used as imaging regions for imaging or options thereof.

The imaging section correction part 47D has a function to overlay the imaging slice sections automatically calculated as the imaging regions with a reference image to display them on the display unit 34 and correct sizes, positions and directions of the imaging slice sections according to information inputted from the input device 33 with referring to the reference image and the imaging slice sections displayed on the display unit 34. For correction of the imaging slice sections, GUI (Graphical User Interface) technique can be used. Then, the latest imaging slice sections can be overlaid and displayed on the reference image in real time.

Then, the operation and action of the magnetic resonance imaging apparatus 20 will be described. Here, an example case of automatically setting imaging regions to a stenosis region of a carotid artery will be described.

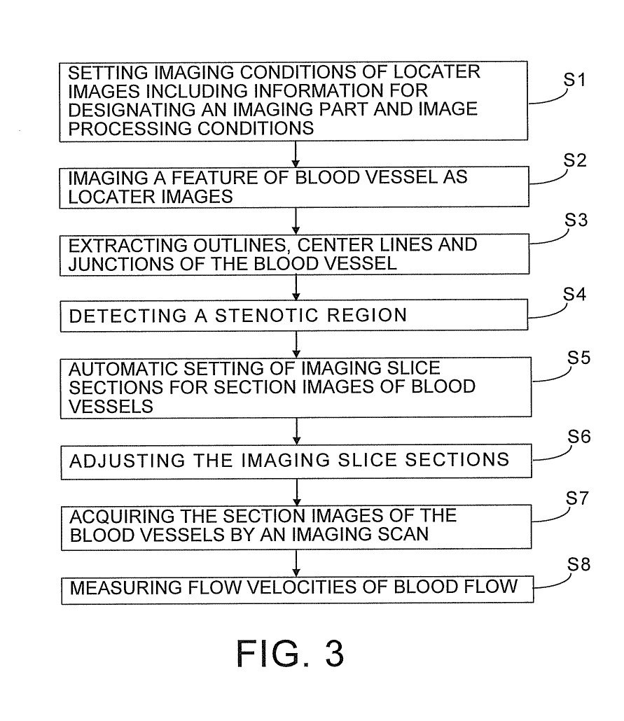

FIG. 3 is a flowchart showing a flow for plaque imaging of a carotid artery of an object P by the magnetic resonance imaging apparatus 20 as shown in FIG. 1.

Firstly, the object P is previously set to the bed 37, and a static magnetic field is generated at an imaging area of the magnet 21 (a superconducting magnet) for static magnetic field excited by the static-magnetic-field power supply 26. Further, the shim-coil power supply 28 supplies current to the shim coil 22, thereby uniformizing the static magnetic field generated at the imaging area. Subsequently, basic section images such as sagittal images, coronal images and axial images are acquired as locator images.

Then, in the step S1, the imaging condition setting unit 40 sets imaging conditions and image processing conditions, for locator images for plaque imaging, including information designating an imaging part. Specifically, an operator refers to a screen for setting the imaging conditions and the image processing conditions displayed on the display unit 34 and operates the input device 33 to input information designating the imaging part in the imaging condition setting unit 40. In this example, the imaging purpose is imaging a carotid artery. Therefore, the imaging part is designated to the carotid artery.

Subsequently, the imaging condition setting unit 40 sets imaging conditions for imaging a feature of the carotid artery as a locator image. Meanwhile, the image processing condition setting unit 41 sets image processing conditions for generating a feature image of the carotid artery as the locator image.

For that purpose, the imaging condition setting unit 40 stores the carotid artery as information designating the imaging part in the condition storage unit 42 while the imaging condition setting unit 40 acquires imaging conditions set for feature imaging of a carotid artery in the past from the condition storage unit 42. Meanwhile, the image processing condition setting unit 41 acquires image processing conditions set for feature imaging of a carotid artery in the past from the condition storage unit 42.

Then, the imaging conditions and the image processing conditions for feature imaging of the carotid artery in the past are displayed for reference on the screen for setting the imaging conditions and the image processing conditions.

The operator edits the imaging conditions and the image processing conditions, as needed, and sets the imaging conditions and the image processing conditions suitable for feature imaging of the carotid artery. More specifically, the imaging conditions and the image processing conditions for imaging multi slice images depicting inner cavity of blood vessels involving a junction part from a gross carotid artery to an inner carotid artery and an outer one are set. As the imaging sections, plural axial sections are set in a region covering the carotid arteries wither referring to the sagittal images acquired as the locator images.

As an imaging method for depicting blood vessels, TOF method can be used. The TOF method is a method for acquiring blood vessel images with use of inflow effect of blood in imaging sections. In the TOF method, blood flowing in the imaging sections after applying a saturation pulse is imaged as a longitudinal relaxation (T1) enhanced image using an FE (field echo) type of pulse sequence with applying the saturation pulse. Therefore, a blood vessel image showing an inner cavity of blood vessels can be acquired by the TOF method.

Accordingly, a three dimensional (3D) or two dimensional (2D) pulse sequence under the TOF method is set as the imaging conditions for example. Note that, blood vessel imaging method, such as FBI (Fresh Blood Imaging) method, other than the TOF method may be used. Further, the image processing conditions, such as subtraction processing, according to the imaging method are set by the image processing condition setting unit 41.

The imaging conditions, for feature imaging of the carotid arteries, set by the imaging condition setting unit 40 are written and stored as the imaging conditions for the locator images in the condition storage unit 42. Meanwhile, the image processing conditions, for feature imaging of the carotid arteries, set by the image processing condition setting unit 41 are written and stored as the image processing conditions for the locator images in the condition storage unit 42.

Next, in the step S2, feature imaging of the carotid arteries is performed for the locator images. Specifically, the operator operates the input device 33 to input instruction for starting imaging to the condition output unit 43. Then, the condition output unit 43 acquires the imaging conditions for feature imaging of the carotid arteries from the condition storage unit 42 and outputs them to the sequence controller 31.

Next, the sequence controller control unit 41 drives the gradient power supply 27, the transmitter 29, and the receiver 30 in accordance with the imaging conditions for feature imaging of the carotid arteries, thereby generating a gradient magnetic field at the imaging area having the set object P, and further generating RF signals from the RF coil 24.

Consequently, the RF coil 24 receives MR signals generated due to nuclear magnetic resonance in the object P. Then, the receiver 30 receives the MR signals from the RF coil 24 and generates raw data which is digital data of NMR signals by A/D conversion subsequently to necessary signal processing. The receiver 30 supplies the MR signals to the sequence controller 31. The sequence controller 31 outputs the MR signals to the computer 32.

Then, the data processing unit 44 of the computer 32 arranges the MR signals, acquired from the sequence controller 31, as k-space data to the k space formed in the k-space data storage unit 45. Subsequently, the data processing unit 44 reconstructs image data by image reconstruction processing of the k-space data read from the k-space data storage unit 45.

Meanwhile, the condition output unit 43 gives the image processing conditions for feature imaging of the carotid arteries acquired from the condition storage unit 42 to the data processing unit 44. Then, the data processing unit 44 performs image processing of the image data according to the image processing conditions acquired from the condition output unit 43. Consequently, feature image data depicting a feature of the carotid arteries is generated.

The feature image data of the carotid arteries generated by the image processing is written and stored in the image database 46. As a result, multi slice image data depicting the feature of the carotid arteries is stored for positioning in the image database 46. Then, acquiring multi slice image data of many slices makes it possible to obtain volume image data of a region including the carotid arteries.

Next, in the step S3, the structural information acquiring part 47A acquires anatomical structural information of the blood vessels based on the multi slice image data as an example of the first image data of the object P. Specifically, the structural information acquiring part 47A acquires the multi slice image data depicting the feature of the carotid arteries from the image database 46 and obtains the structural information including blood vessel center lines and junctions of the carotid arteries by analytic processing

FIG. 4 is a view showing an example of center line, outline and junction of carotid arteries obtained by the structural information acquiring part 47A shown in FIG. 2.

In FIG. 4, the solid lines represent inner wall of the blood vessels consisting of the carotid arteries, the dotted line represents the center line of the gross carotid, the chain line represents the center line of the outer carotid artery and the chain double-dashed line represents the center line of the inner carotid artery respectively. As shown in FIG. 4, the carotid arteries have a structure branching into the inner carotid artery and the outer carotid artery from the gross carotid artery. The structural information acquiring part 47A extracts center lines, branching positions and a outline of such branching blood vessels.

The outline of blood vessels can be extracted as borders of an area showing signal values corresponding to that from blood flow in the multi slice image data by known processing such as edge extraction processing. Note that, the signal values from the blood flow can be estimated based on the imaging conditions and the image processing conditions.

The center lines of blood vessels can be extracted by known arbitrary data processing such as processing which connects barycentric positions of regions corresponding to insides of the blood vessels on plural 2D sections, i.e, between the 2D sections. When the center lines of the blood vessels have been obtained, a position at which a center line of blood vessel branches can be obtained as a position of junction of blood vessels.

Further, it becomes possible to classify the blood vessels into the gross carotid artery, the inner carotid artery and the outer carotid artery of which junction is each end point by an arbitrary method such as pattern matching with known anatomical information of human body. As a result of the segmentation processing for the respective branches, each blood vessel inner cavity region of the gross carotid artery, the inner carotid artery and the outer carotid artery can be specified.

Next, in the step S4, the abnormal part detection part 47B detects an abnormal region based on the structural information acquired by the structural information acquiring part 47A. Specifically, the abnormal part detection part 47B detects a stenosis region of the carotid arteries as the abnormal region by data analysis processing of the structural information such as the center lines, the outline and the junctions of the carotid arteries.

As a processing method for detecting a stenosis region of blood vessels, an arbitrary method can be used. For example, a normal blood vessel has a constant internal diameter or changes with a constant rate to thin down toward the end. On the contrary, a stenosis part has a locally thin internal diameter of a blood vessel.

Accordingly, a stenosis part can be detected analytically by obtaining internal diameters of blood vessels on sections normal to the center lines of the blood vessels at a predetermined interval to estimate a variation in internal diameter of a blood vessel in the center line direction. For example, a point on a center line at which an internal diameter of blood vessel shows a local minimum value is detected. Then, a range in which the internal diameter of the blood vessel is smaller than a predetermined size in the vicinity of the local minimum value can be considered as a stenosis region.

Note that, a threshold may be set to a stenosis rate so that an abnormal part can be detected as an area showing the stenosis rate not lower than a predetermined value. The stenosis rate can be specified as a ratio between a section area of a blood vessel at a position at which the internal diameter of the blood vessel shows a local minimum value and that at a position at which the internal diameter of the blood vessel recovers up to a predetermined sizes.

Further, the multi slice image data used for acquiring the structural information of the blood vessels may be referred to for detecting an abnormal part according to a detection algorithm of the abnormal part, as needed.

On the other hand, when no abnormal part has been detected, detection result information indicating that no abnormal part has been detected is generated by the abnormal part detection part 47B.

Next, in the step S5, the imaging section calculation part 47C automatically sets sizes, positions and directions of imaging slice sections for imaging of blood vessel section images by data processing of the stenosis region detected as the abnormal part.

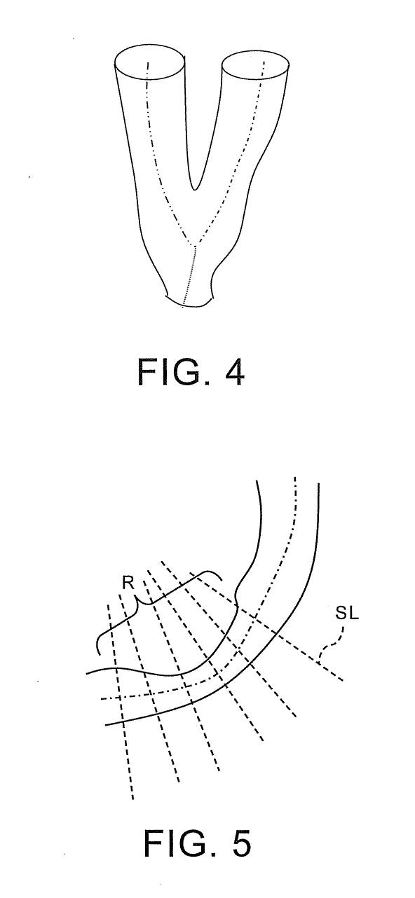

FIG. 5 is a view for explaining the first example method of setting imaging slice sections by the imaging section calculation part 47C shown in FIG. 2.

In FIG. 5, the solid lines represent inner wall of a blood vessel, the chain line represents the center line of the blood vessel and the dashed lines represent positions of imaging slice sections respectively. As shown in FIG. 5, the imaging section calculation part 47C can automatically set imaging slice sections SL each having an appropriate size and direction at appropriate positions according to a stenosis region R. FIG. 5 shows an example of setting plural slice sections SL so that the center of FOV (field of view) on each slice section SL becomes on the center line of the blood vessel and each slice section SL becomes normal to the center line of the blood vessel. Further, the slice sections SL, of which number and sizes are set to be those necessary for covering the stenosis region R, are arranged at a constant interval along the center line of the blood vessel.

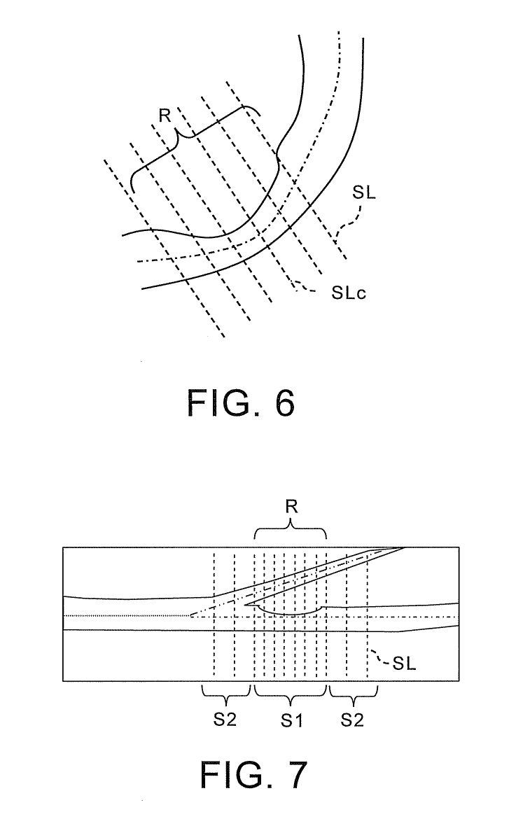

FIG. 6 is a view for explaining the second example method of setting imaging slice sections by the imaging section calculation part 47C shown in FIG. 2.

In FIG. 6, the solid lines represent inner wall of a blood vessel, the chain line represents the center line of the blood vessel and the dashed lines represent positions of imaging slice sections respectively. As shown in FIG. 6, slice sections SLc may be set so as to be normal to the center line at a position on the center line at which the internal diameter of the blood vessel becomes the minimum in the stenosis region R. In addition, a necessary number of slice sections may be set so as to be parallel to the set slice sections SLc. The number of the slice sections SL is set to be one necessary for covering the stenosis region R. The interval of the slice sections SL is set to be constant.

As shown in FIG. 5 and FIG. 6 as examples, at least one section normal to a center line of blood vessel in carotid arteries can be indicated as an imaging region according to detection result of an abnormal region. Setting the imaging slice sections as shown in FIG. 6 is advantageous for reducing time necessary for imaging of blood vessel section images. Meanwhile, setting the imaging slice sections as shown in FIG. 5 makes it possible to acquire blood vessel section images of which all are normal to the blood vessel.

FIG. 7 is a view for explaining the third example method of setting imaging slice sections by the imaging section calculation part 47C shown in FIG. 2.

As shown in FIG. 7, the imaging slice sections SL can be also indicated on image data obtained by a desired image processing of feature image data of blood vessels. In the example shown in FIG. 7, the imaging slice sections SL are indicated on a SPR (Stretched Curved Multiple Planer Reconstruction) image.

Further, first plural imaging sections corresponding to inside of an abnormal region and the second plural imaging sections corresponding to outside of the abnormal region may be indicated. In this case, an interval of the first plural imaging sections may be set to be narrower than that of the second imaging sections. Specifically, the first plural imaging sections can be automatically set so as to cover the abnormal region. Meanwhile, the second plural imaging sections can be automatically set for observing features outside of a region to which the first plural imaging sections have been set. In this case, the interval of the first plural imaging sections can be set to be narrow for observing the abnormal part while that of the second plural imaging sections can be set to relatively large for observing the features. Consequently, a time required for imaging can be reduced while images necessary for diagnosis are acquired. As a concrete example, the interval of the second imaging slice group S2 arranged outside of the first imaging slice group S1 may be set to be larger than that of the first imaging slice group S1 arranged so as to cover the stenosis region R as shown in FIG. 7.

Note that, when no stenosis region as abnormal part has been detected, the imaging section calculation part 47C may be set imaging slice sections in a region involving a junction part of blood vessels automatically. On the contrary, though a stenosis region has been detected, the imaging section calculation part 47C may be set imaging slice sections in a region involving a junction part of blood vessels automatically in addition to the stenosis region. Further, one or both of the structural information of the carotid arteries and the multi slice image data may be referred to for automatically setting of imaging slice sections, as needed.

The imaging slice sections set as described above can be displayed as options for imaging slice sections for imaging on the display unit 34. Specifically, the imaging section calculation part 47C indicates imaging regions, according to detection result of the abnormal region by the abnormal part detection part 47B, to the operator through the display unit 34. Then, the operator can correct sizes, positions and directions of the imaging slice sections, as needed.

In that case, in the step S6, the imaging section correction part 47D adjusts the imaging slice sections according to information inputted from the input device 33. For adjustment, the imaging slice sections can be displayed on the display unit 34 in various methods.

For example, the imaging slice sections can be overlaid and displayed as a region of interest (ROI) on a 3D image, such as a VR (volume rendering) image, a MIP (maximum intensity projection) image, a CPR (Curved Multiple Planer Reconstruction) image or a SPR image, depicting feature of blood vessels. Each 3D image can be generated from the multi slice image data of the carotid arteries acquired by feature imaging. When a 3D image of blood vessels is used as a reference image, the imaging slice sections can be displayed as an overhead view on the display unit 34

Note that, a CPR image is derived by image reconstruction processing which transforms a curved surface into a plane. When a CPR image is generated, a blood vessel traveling three dimensionally can be depicted on a single plane. Meanwhile, a SPR image is derived by image reconstruction processing which transforms a curve of a CPR image into a line. When a SPR image is generated, a blood vessel traveling three dimensionally can be depicted on a single line. For example, when a SPR image is generated by transforming a traveling direction of a certain blood vessel into the horizontal direction, directions of sections of the blood vessel become the vertical direction.

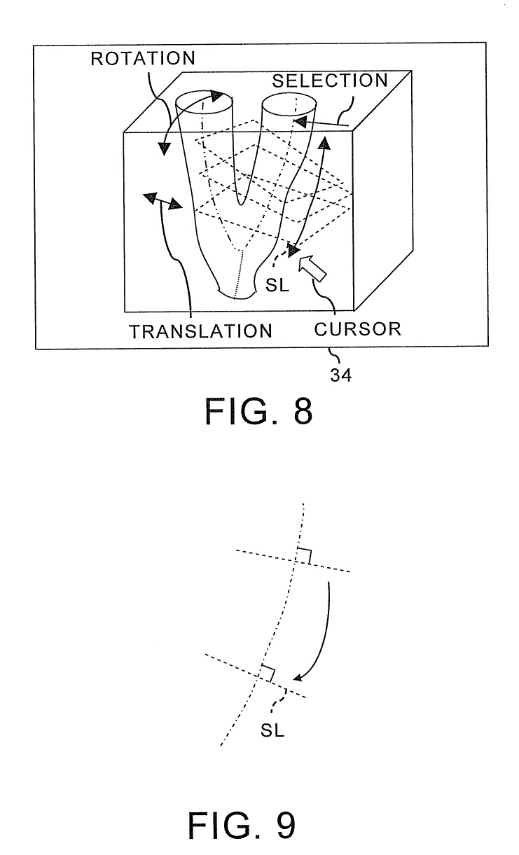

FIG. 8 is a view showing an example of overlaying and displaying imaging slice sections on a VR image of blood vessels for adjusting the imaging slice sections by the imaging section correction part 47D shown in FIG. 2.

In FIG. 8, the solid lines represent inner wall of blood vessels consisting of the carotid arteries, the dotted line represents the center line of the gross carotid artery, the dashed line represents the center line of the outer carotid artery, the chain double-dashed line represents the center line of the inner carotid artery and the broken lines represent positions of the imaging slice sections respectively. As shown in FIG. 8, the imaging slice sections automatically set by the imaging section calculation part 47C can be displayed as a ROI for positioning together with a VR image depicting a feature of blood vessels on the display unit 34.

The operator can select a desired imaging slice section with the input device 33 such as a mouse and correct the selected imaging slice section by operating the input device 33. For example, drag and drop of a desired imaging slice section can move the desired imaging slice section along the center line of the blood vessel. Alternatively, an imaging slice section can be moved on a same plane to shift the center of the imaging slice section from the center line of the blood vessel.

In addition, an imaging slice section can be rotated three dimensionally, expanded and reduced. Specifically, the imaging section correction part 47D can rotate an imaging section with fixing the center of the imaging section, to be an imaging region according to detection result of the abnormal region, on the center line of the blood vessel, for adjusting the imaging section, according to information inputted from the input device 33. Further, with fixing the center of an imaging section on the center line of the blood vessel, the imaging section can be expanded and reduced.

As described above, parallel movement, rotation, expansion and reduction of a desired imaging slice section can be performed. Especially, when an imaging slice section is moved along the center line of the blood vessel, the imaging section correction part 47D can automatically adjust the direction of the imaging slice section so as to be normal to the center line of the blood vessel constantly.

FIG. 9 is a view for explaining a method for moving an imaging slice section along the center line of the blood vessel by the imaging section correction part 47D shown in FIG. 2.

In FIG. 9, the dashed line represents a center line of a blood vessel, the broken lines represent imaging slice sections. As shown in FIG. 9, when an imaging slice section is moved along the center line of the blood vessel with the input device 33 such as a mouse, the direction and position of the imaging slice section is automatically adjusted by the imaging section correction part 47D so that the direction of the imaging slice section is constantly normal to the center line of the blood vessel and the center of the imaging slice section lies constantly on the center line of the blood vessel.

Consequently, an operator can finish movement of an imaging slice section along a center line of a blood vessel by one operation of movement of the imaging slice section along a curve although two operations of parallel movement and rotation of the imaging slice section have been conventionally required. Therefore, appropriate imaging slice sections can be set easily.

Further, in the VR image shown in FIG. 8, an imaging slice section can be added and deleted. For example, when a point on the center line of the blood vessel is designated for adding an imaging slice section by the input device 33 such as a mouse, the imaging section correction part 47D automatically sets an imaging slice section, of which center is the designated point, normal to the center line of the blood vessel. Alternatively, when an arbitrary point is designated and an arbitrary imaging slice section is selected by the input device 33, the imaging section correction part 47D automatically sets an imaging slice section parallel to the selected imaging slice section and passing through the designated point.

Further, in the VR image shown in FIG. 8, when a branch of blood vessels is selected, the selected blood vessel may be displayed as a CPR image or a SPR image.

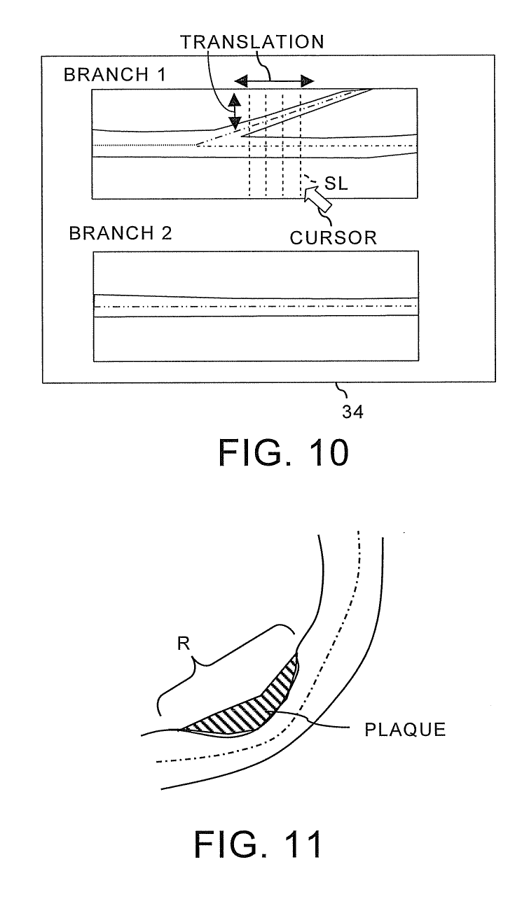

FIG. 10 is a view showing an example of overlaying and displaying imaging slice sections on a SPR image of blood vessels for adjusting the imaging slice sections by the imaging section correction part 47D shown in FIG. 2.

In FIG. 10, the solid lines represent inner wall of blood vessels consisting of the carotid arteries, the dotted line represents the center line of the gross carotid artery, the dashed line represents the center line of the outer carotid artery, the chain double-dashed line represents the center line of the inner carotid artery and the broken lines represent positions of imaging slice sections respectively. As shown in FIG. 10, each branch of blood vessel can be displayed as a SPR image. In the example shown in FIG. 9, a branch 1 and a branch 2 are displayed as SPR images respectively. In each SPR image, the attended branch is displayed linearly while the other branches are displayed in oblique directions.

Further, the imaging slice sections automatically set by the imaging section calculation part 47C can be overlaid and displayed on a SPR image. Then, an operator can move, add and delete an imaging slice section and alter a size of an imaging slice section by operating the input device 33.

On a SPR image, a center line of blood vessel becomes a line and imaging slice sections are constantly orthogonal to the center line of the blood vessel. Therefore, by moving a imaging slice section in the horizontal direction, the imaging slice section can be moved along the center line of the blood vessel with keeping the direction normal to the center line of the blood vessel. Further, by moving an imaging slice section in the vertical direction, the imaging slice section can be moved on the same plane. That is, the center of the imaging slice section can be shifted from the center line of the blood vessel.

Further, by designating an arbitrary point on the center line of the blood vessel, an imaging slice section, of which center is the designated point on the center line of the blood vessel, normal to the center line of the blood vessel can be added. Furthermore, when an arbitrary point on a SPR image is selected, an imaging slice section, of which center is the selected point, normal to the center line of the blood vessel can be added.

FIG. 8 and FIG. 10 shows examples of displaying the imaging slice sections on the VR image and the SPR image respectively. However, imaging slice sections may be also displayed on a MIP image or a CPR image similarly. Note that, when imaging slice sections are displayed on a 2D image or a CPR image, an image as shown in FIG. 5 or FIG. 6 is derived. Then, movement, rotation, expansion, reduction, addition and deletion of an imaging slice section can be performed so as to be normal to a center line of a blood vessel with operation of the input device 33 by an operator similarly to a case of displaying the imaging slice sections on a VR image or a SPR image.

That is, MIP image data or VR image data of carotid arteries can be displayed together with imaging regions according to detection result of an abnormal region on the display unit 34. Then, the imaging sections, as imaging regions according to the detection result of the abnormal region, can be adjusted so as to be normal to a blood vessel center line of the carotid arteries according to information inputted from the input device 33.

Further, branches of the carotid arteries selected with referring to the MIP image data or the VR image data of the carotid arteries by operation of the input device 33 can be displayed as a CPR image or a SPR image together with the imaging regions according to the detection result of the abnormal region on the display unit 34. Then, the imaging sections as the imaging regions according to the detection result of the abnormal region can be adjusted so as to be normal to a blood vessel center line of the carotid arteries according to information inputted from the input device 33

Note that, kinds of operations by the input device 33, such as click, drag and drop by a mouse, can be assigned to various contents arbitrary in advance.

The imaging regions manually adjusted with operation of the input device 33 as described above are displayed on the display unit 34 in real time. Then, the imaging regions after the adjustment by the imaging section correction part 47D are given to the imaging condition setting unit 40 as setting information of imaging regions for imaging.

Meanwhile, when manual adjustment is not necessary, the imaging slice sections automatically set by the imaging section calculation part 47C are given to the imaging condition setting unit 40 as setting information of imaging regions for imaging. In this case, an input of confirmation information by operation of the input device 33 may be used as a trigger.

When imaging regions for imaging have been set based on the imaging regions according to the detection result of the abnormal region, it becomes possible to acquire the second image data of the object P by imaging of the set imaging regions. As examples of the second image data include image data for estimating a plaque nature with regard to a blood vessel and image data for measuring flow velocities of blood flow. Therefore, at least one of plaque imaging of a blood vessel and acquisition of image data for measuring flow velocities of blood flow can be performed. Here, a case of acquiring image data for measuring flow velocities of blood flow subsequently to plaque imaging of carotid arteries will be described.

In that case, in the step S7, plaque imaging of the carotid arteries is performed. Specifically, the imaging regions supplied from the imaging region setting unit 47 are set to imaging regions for the plaque imaging by the imaging condition setting unit 40 at first. In addition, the other imaging conditions for the plaque imaging are set by the imaging condition setting unit 40. Meanwhile, image processing conditions for the plaque imaging are set by the image processing condition setting unit 41.

FIG. 11 is a view showing a plaque region to be a target of the plaque imaging by the magnetic resonance imaging apparatus 20 shown in FIG. 1.

In FIG. 11, the solid lines represent inner wall of a blood vessel, the dashed line represents a center line of the blood vessel and the shaded area represents an assumed plaque region respectively. As shown in FIG. 11, it is estimated that a plaque region exists between the inner wall and the outer wall of the blood vessel in the stenosis region R. Accordingly, section images of the blood vessel crossing the plaque region are acquired by the plaque imaging.

The TOF method, which depicts blood flow for acquiring locator images, cannot image the plaque region clearly. Accordingly, imaging conditions for enhancing MR signals from the plaque are set for the plaque imaging so that a plaque nature can be estimated. Specifically, by imaging conditions such as T1 enhancing conditions or transverse relaxation (T2) enhancing conditions, images can be acquired with various contrasts. For that reason, imaging conditions are set according to a plaque nature such as a stable plaque, an unstable plaque, a lipid core or a lipid core with bleed.

In addition, a resolution higher than that for locator image data is set as an imaging condition for the plaque imaging by the imaging condition setting unit 40. For that purpose, an appropriate matrix size is set as an imaging condition. Further, all or some of the RF coils 24 for reception of MR signals are changed to keep a reception sensitivity of MR signals necessary for the plaque imaging, as needed.

Then, an imaging scan is performed in a flow similar to that for acquiring the locator images. Consequently, image data of blood vessel sections in a part involving the plaque is acquired. The acquired image data of the blood vessel sections is stored in the image database 46. Then, the image data of the blood vessel sections can be displayed on the display unit 34 for observation.

The imaging slice sections for the plaque imaging had been automatically set by the imaging region setting unit 47 so as to be normal to the center line of the blood vessel in the stenosis region automatically extracted from the locator image data. Therefore, image data of the blood vessel sections useful for diagnosis can be obtained.

Next, in the step S8, data acquisition for estimating blood flow velocities is performed. Specifically, imaging conditions and image processing conditions of image data for measuring flow velocities of blood flow are set by the imaging condition setting unit 40 and the image processing condition setting unit 41 respectively.

As image data for measuring flow velocities of blood flow, cine image data acquired by the PS flow method is preferable. For that reason, the imaging condition setting unit 40 sets imaging conditions which set imaging slice sections, set by the imaging region setting unit 47, to imaging regions under the PS flow method.

Then, MR signals are acquired in a flow similar to that in the plaque imaging of the carotid arteries to generate cine image data for measuring flow velocities of blood flow. Further, flow velocities of blood flow are measured based on the cine image data.

Blood flows slowly in the vicinity of blood vessel wall and has the maximum velocity in the vicinity of the center in the blood vessel. Specifically, blood flow has a distribution of flow velocities in blood vessel. Therefore, when an imaging slice section is set as a ROI to measure a mean velocity of blood flow, a value of the mean flow velocity varies depending on a size of the ROI and a relative position to the blood vessel of the ROI.

Specifically, when the ROI is set to be normal to a traveling direction of blood vessel, i.e., a direction along a flow of blood, it becomes possible to measure flow velocities of the blood flow with the highest accuracy. When the ROI is small and covers only the vicinity of the center on the blood vessel section, velocities of the blood flow are overestimated while a quantity of the blood flow is underestimated. On the contrary, when the ROI is set to be excessively large compared to a size of the blood vessel section, velocities of the blood flow are underestimated while a quantity of the blood flow becomes a more accurate value.

On the other hand, the imaging region setting unit 47 sets the imaging slice sections having appropriate sizes according to a size of the stenosis region of blood vessel and normal to the center line of the blood vessel. Consequently, the cine image data acquired by cine imaging becomes image data appropriate for measuring flow velocities of blood. Therefore, the flow velocities of blood can be measured with higher accuracy.

As described above, the magnetic resonance imaging apparatus 20 is an apparatus configured to be able to acquire anatomical structural information by image processing of locator images and automatically indicate or set appropriate imaging regions according to a morphological abnormal region detected by analytic processing based on the anatomical structural information

For example, in case of plaque imaging of carotid arteries, center lines, an outline of inner walls and positions of junctions with regard to blood vessels consisting of the carotid arteries are specified as anatomical structural information based on volume image data of the blood vessels, and subsequently, a stenosis region is detected as an abnormal region in feature. Then, sections covering the stenosis region and normal to the traveling direction of the blood vessel are automatically indicated or set as imaging regions.

Such indication and setting of imaging regions can be performed to a desired imaging target.

For example, center line information of an imaging target can be acquired based on the first image data of an object P, such as locator image data, and then, a pick of an abnormal region and orthogonal sections for imaging the pick of the abnormal region can be indicated based on the center line information. Consequently, imaging sections can be confirmed by referring to the indicated orthogonal sections. Then, the second image data of the object P can be acquired by imaging f the imaging sections. When the imaging target is a blood vessel, a stenosis rate along a center line of the imaging target can be also calculated to acquire a pick of an abnormal region based on the stenosis rate.

Concrete examples other than a blood vessel include imaging of a spine. Specifically, in case of setting imaging slice sections so as to be normal to the longitudinal direction of the spine and cover an abnormal part of the spine, the above mentioned method for automatically indicating imaging regions can be also applied. For example, when herniated intervertebral discs consist of an abnormal part, a region in which the herniated intervertebral discs occur in the spine can be specified as the abnormal part to automatically set imaging slice sections appropriate for the specified herniated intervertebral discs region.

Extraction of the center line of the spine can be performed by image processing, including removing noise by smoothing processing and specifying an outline of the spine by edge extraction processing, of feature image data of the spine acquired according to imaging conditions for depicting a feature of the spine clearly. By such image processing, outlines of respective vertebral bones and interspinal disks can be extracted. Subsequently, a curve obtained by connecting the respective barycenters of the extracted vertebral bones and interspinal disks smoothly by an interpolation such as a spline interpolation can be considered as the center line of the spine.

Extraction of the abnormal part of the spine can be performed by determination whether each barycenter of the interspinal disks is far from the extracted center line of the vertebral bones beyond a predetermined threshold, or , whether each outline of the interspinal disks protrudes from an outline of the extracted vertebral bones beyond a predetermined threshold, for example.

Then, by the threshold processing, a herniated intervertebral discs region of the spine or a part suspected of herniated intervertebral discs can be detected as an abnormal region or a pick of abnormal region. Then, the abnormal part can be specified more steadily by inputting confirmation information or selecting an interspinal disk by operation of the input device 33.

In addition to such automatic setting of imaging slice sections according to an abnormal part, the magnetic resonance imaging apparatus 20 is configured to display the imaging regions set automatically once on a 3D image such as a MIP image, a VR image, a CPR image or a SPR image as an overhead view so that the imaging regions can be adjusted manually by an operator with appropriate restrictions according to anatomical structural information. For example, imaging regions can be adjusted with a restriction that imaging slice sections are constantly normal to a traveling direction of a blood vessel or a spine.

Therefore, the magnetic resonance imaging apparatus 20 makes it possible to set imaging regions easily in case of imaging of sections of tissue having a complex structure or a blood vessel. Specifically, an operator becomes possible to omit observation of locator images and setting of imaging regions involving a stenotic part. Especially, the magnetic resonance imaging apparatus 20 is useful for setting of imaging regions for blood vessel section images necessary for estimation of a nature of plaque and/or measuring flow velocities of blood in a stenotic part of a branching and/or curved blood vessel such as a carotid artery.

In addition, it is possible to set sections normal to a traveling direction of a curved blood vessel such as a carotid artery as imaging regions easily. Consequently, flow velocities of blood can be measured with high accuracy. As a result, a throughput in an entire examination can be improved with keeping accuracy in the examination.

Further, an operator can display imaging regions on a 3D image such as a MIP image to edit the imaging regions manually. Especially, imaging slice sections can be easily adjusted according to a traveling direction of a blood vessel or a spine. Adjustment of imaging regions through a MIP image and the like is effective when a disease such as stenosis is found on a MRA image and it is important to grasp a medical condition in more detail. More specifically, adjustment of imaging regions through a MIP image and the like is effective for a case of acquiring FS-BB (flow-sensitive black-blood) images f a head, examination of aneurism in an abdomen and the like as well as plaque imaging of a carotid artery.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. A magnetic resonance imaging apparatus comprising:
a structural information acquisition unit configured to acquire anatomical structural information based on first image data of an object;
an abnormal part detection unit configured to detect an abnormal region based on the structural information;
an imaging region setting unit configured to indicate an imaging region according to a detection result of the abnormal region; and
an imaging unit configured to acquire second image data of the object by imaging of an imaging region set based on the imaging region according to the detection result of the abnormal region.

2. A magnetic resonance imaging apparatus of claim 1,
wherein said structural information acquisition unit is configured to acquire structural information involving a center line and a junction of blood vessels consisting of carotid arteries,
said abnormal part detection unit is configured to detect a stenosis region of the carotid arteries as the abnormal region, and
said imaging region setting unit is configured to indicate a section normal to the center line of the carotid arteries as the imaging region according to the detection result of the abnormal region, further comprising:
an imaging section correction unit configured to display a branch of the carotid arteries together with the imaging region according to the detection result of the abnormal region as a Curved Multiple Planer Reconstruction image or a Stretched Curved Multiple Planer Reconstruction image on a display unit to adjust an imaging section consisting of the imaging region according to the detection result of the abnormal region so as to be normal to the center line of the carotid arteries according to information inputted from an input device, the branch being selected by an operation of the input device or another input device with referring to maximum intensity projection image data or volume rendering image data of the carotid arteries.

3. A magnetic resonance imaging apparatus of claim 1,
wherein said structural information acquisition unit is configured to acquire structural information involving a center line and a junction of blood vessels consisting of carotid arteries,
said abnormal part detection unit is configured to detect a stenosis region of the carotid arteries as the abnormal region, and
said imaging region setting unit is configured to indicate a section normal to the center line of the carotid arteries as the imaging region according to the detection result of the abnormal region,
further comprising:
an imaging section correction unit configured to display maximum intensity projection image data or volume rendering image data of the carotid arteries together with the imaging region according to the detection result of the abnormal region on a display unit to adjust an imaging section consisting of the imaging region according to the detection result of the abnormal region so as to be normal to the center line of the carotid arteries according to information inputted from an input device.

4. A magnetic resonance imaging apparatus of claim 1,
wherein said structural information acquisition unit is configured to acquire structural information involving a center line and a junction of blood vessels consisting of carotid arteries,
said abnormal part detection unit is configured to detect a stenosis region of the carotid arteries as the abnormal region, and
said imaging region setting unit is configured to indicate a section normal to the center line of the carotid arteries as the imaging region according to the detection result of the abnormal region.

5. A magnetic resonance imaging apparatus of claim 1,
wherein said structural information acquisition unit is configured to acquire structural information of a blood vessel or a spine.

6. A magnetic resonance imaging apparatus of claim 1,
wherein said structural information acquisition unit is configured to acquire at least one of a center line and an inner cavity of a blood vessel as the structural information.

7. A magnetic resonance imaging apparatus of claim 1,
wherein said structural information acquisition unit is configured to acquire structural information of a blood vessel, and
said abnormal part detection unit is configured to detect a stenosis region of the blood vessel as the abnormal region.

8. A magnetic resonance imaging apparatus of claim 1,
wherein said structural information acquisition unit is configured to acquire structural information of a spine, and
said abnormal part detection unit is configured to detect a herniated intervertebral discs region of the spine as the abnormal region.

9. A magnetic resonance imaging apparatus of claim 1,
wherein said imaging unit is configured to perform at least one of plaque imaging of a blood vessel and acquisition of image data for measuring a flow velocity of blood.

10. A magnetic resonance imaging apparatus of claim 6, further comprising:
an imaging section correction unit configured to rotate an imaging section as the imaging region according to the detection result of the abnormal region with fixing a center of the imaging section on the center line of the blood vessel according to information inputted from an input device.

11. A magnetic resonance imaging apparatus of claim 6, further comprising:
wherein said imaging region setting unit is configured to indicate first plural imaging sections corresponding to an inside of the abnormal region and second plural imaging sections corresponding to an outside of the abnormal region, an interval between the first plural imaging sections being narrower than an interval between the second plural imaging sections.

12. A magnetic resonance imaging apparatus comprising:
a center line information acquisition unit configured to acquire center line information of an imaging target based on first image data of an object;
an indication unit configured to indicate a pick of an abnormal region and an orthogonal section for imaging the pick of the abnormal region based on the center line information;
a confirmation unit configured to confirm an imaging section with referring to the indicated orthogonal section; and
an imaging unit configured to acquire second image data of the object by imaging of the imaging section.

13. A magnetic resonance imaging apparatus of claim 12,
wherein said indication unit is configured to calculate a stenosis rate along a center line of the imaging target to acquire a pick of the abnormal region based on the stenosis rate.

14. A magnetic resonance imaging method comprising:
acquiring anatomical structural information based on first image data of an object;
detecting an abnormal region based on the structural information;
indicating an imaging region according to a detection result of the abnormal region; and
acquiring second image data of the object by imaging of an imaging region set based on the imaging region according to the detection result of the abnormal region.
